(19) United States
(12) Patent Application Publication
(10) Pub. No.: US 2007/0155962 A1
(43) Pub. Date: Jul. 5, 2007

(54) Process for preparing titanyl phthalocyanine

(76) Inventors: Ling Lu, Taoyuan Hsien (TW); Chang-Yu Chou, Tainan Hsien (TW); Chang-Li Cheng, Tainan City (TW); Po-Han Huang, Tainan City (TW); Yuan-Szu Chang, Kaohsiung Hsien (TW); Nien-Szu Huang, Tainan Hsien (TW); Shyue-Ming Jang, Hsinchu (TW)

Correspondence Address: Rabin & Berdo, P.C., Suite 500, 1101 14th Street, Washington, DC, 20005, US

(21) Appl. No.: 11/322,077

(22) Filed: Dec. 30, 2005

(51) Int. Cl. C07D 487/22

(52) U.S. Cl. 540/143

(57) ABSTRACT

A process for preparing titanyl phthalocyanines in one reaction reactor includes the reaction of titanium tetrachloride or titanium trichloride and o-phthalodinitrile in an organic solvent such as 1-chloronaphthalene in the presence of a molecular sieve as a promoter followed by hydrolysis resulting in titanyl phthalocyanines. The prepared titanyl phthalocyanines is usable as a high-quality charge generating material and can be used as a charge generating layer in an organic photoconductor drum.

### BACKGROUND OF THE INVENTION

1. Field of the Invention

The present invention relates to a process for preparing titanyl phthalocyanine (TiOPc) and more particularly to a process that prepares titanyl phthalocyanine in the presence of a molecular sieve as a promoter and employs a synthesizing reaction and hydrolysis in one reaction reactor.

2. Related Prior Art

Titanyl phthalocyanines are very important chemical materials because of their good coloration and are widely used in conventional blue dyes.

Recently, titanyl phthalocyanines were found to have good electrical properties so they are used as efficient charge generating materials that can be used in organic electroluminescent devices, light emitting diodes and organic photoconductor drums.

The technology for preparing a series of titanyl phthalocyanines used as charge generating materials has been disclosed in U.S. Pat. Nos. 4,777,251, 5,164,493, 5,420,268, Japanese Patents JP11279430, JP3258860, JP3291281, JP3199268, JP3021669, JP4193882, JP4246473, JP4193883, JP4266972, JP2169671, JP4277563, JP6200175, JP8027392, JP8176457, JP9104829, JP9165527, JP2000026467, European Patent EP0399430 and People's Republic of China Patent 95103457X.

Generally, conventional processes for preparing titanyl phthalocyanine comprise sequentially reacting o-phthalodinitrile with titanium tetrachloride in an organic solvent at a temperature of 170° C. to 300° C., filtering off the resulting intermediate product dichlorotitanium phthalocyanine and performing hydrolysis as disclosed, for example, in U.S. Pat. No. 4,777,251. Further, a process for the preparation of titanyl phthalocyanine with reactants corresponding to those used in conventional processes is disclosed in U.S. Pat. No. 5,164,493, which comprises the reaction of titanium tetraalkoxide with phthalonitrile and diiminoisoindoline in a solvent. To obtain titanyl phthalocyanine in a short time, JP3021669 discloses a process in which phthalodinitrile is reacted with titanium tetrachloride in an alcohol-based solvent (such as n-amyl alcohol) in the presence of a proton transfer type reaction promoter (e.g. 1,8-diazabicyclo[5,4,0]unde-7-cene) by heating under reflux. Other processes such as using phenol as a promoter or using corresponding reactants (e.g. replacing o-phthalodinitrile with o-phthalimide) are disclosed in the aforementioned documents.

Although titanyl phthalocyanines can be obtained by processes disclosed in the aforementioned documents, said processes have some drawbacks. For example, high temperature (170° C.-300° C.) is required for the conventional processes disclosed in U.S. Pat. No. 4,777,251, corresponding reactants (e.g. phthalimide) utilized are more expensive, and use of phenol as a promoter would pollute the environment since phenol is harmful. In addition, all processes described in the prior art require an extra step to separate and purify the intermediate product (dihalotitanium phthalocyanine or dialkoxytitanium phthalocyanine) before hydrolyzing the intermediate product. The extra step makes the process complicated and lowers the product yield. Furthermore, formation of byproducts impede the main reaction due to the existence of water so a long time is required to finish the reaction (10-24 hours) and obtain the object product.

### SUMMARY OF THE INVENTION

Therefore, an objective of the present invention is to provide a quick, high-yield process for preparing high-purity titanyl phthalocyanine.

Accordingly, a process for preparing titanyl phthalocyanine in accordance with the present invention comprises effecting a synthesizing reaction of titanium tetrachloride or titanium trichloride with o-phthalodinitrile in an organic solvent in the presence of a molecular sieve as a promoter for about 3 to 4 hours to obtain dichlorotitanium phthalocyanine, and hydrolyzing the resulting dichlorotitanium phthalocyanine.

With reference to Scheme (I) below, a process for preparing titanyl phthalocyanine (4) comprises effecting a synthesizing reaction of titanium tetrachloride or titanium trichloride (1) with o-phthalodinitrile (2) in a 1-chloronaphthalene solvent (3) in the presence of a molecular sieve as a promoter for about 3 to 4 hours resulting in dichlorotitanium phthalocyanine, filtering the resulting dichlorotitanium phthalocyanine, adding an equal amount of the previous molecular sieve as additional promoter and hydrolyzing the dichlorotitanium phthalocyanine.

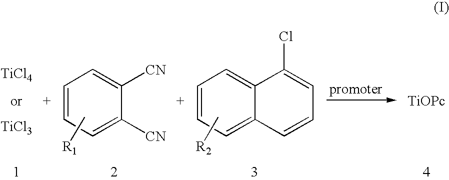

where:

R₁ and R₂ are independently selected from a group consisting of hydrogen, alkyl (C₁-C₅), alkoxy (C₁-C₅) and phenyl;

a stoichmetric ratio of titanium tetrachloride (1): o-phthalodinitrile (2): 1-chloronaphthalene (3) is 1:4.2±0.2:11.1±0.5 or titanium trichloride (1): o-phthalodinitrile (2): 1-chloronaphthalene (3) is1:3.42±0.2:9.52±0.5; and

the weight ratio of the titanium tetrachloride or titanium trichloride to the molecular sieve is from 1:0.5 to 1:5.0.

Further benefits and advantages of the present invention will become apparent after a careful reading of the detailed description in company with the drawings.

### BRIEF DESCRIPTION OF THE DRAWINGS

FIG. 1A˜D is a schematic flow chart of the process for preparing titanyl phthalocyanine in accordance with the present invention.

### DETAILED DESCRIPTION OF THE INVENTION

A process for preparing titanyl phthalocyanine in accordance with the present invention comprises effecting a synthesizing reaction of o-phthalodinitrile with titanium tetrachloride or titanium trichloride in an organic solvent in the presence of a molecular sieve as a promoter for 3 to 4 hours and hydrolyzing the resulting intermediate product dichlorotitanium phthalocyanine to obtain titanyl phthalocyanine. The process performs the synthesizing reaction and hydrolysis in one reaction reactor without separating the intermediate product so the process is simplified and the manufacturing cost is reduced. The molecular sieve dehydrates during the synthesizing reaction and accelerates removal of chlorine ions from the dichlorotitanium phthalocyanine during hydrolysis so the preparation time is reduced. Further, the molecular sieve can be recycled and used repeatedly so waste promoter is significantly reduced, which reduces the cost for handling waste and virtually eliminates pollution of the environment from waste promoter.

Any stoichiometric ratio of titanium tetrachloride or titanium trichloride to o-phthalodinitrile may be employed in the reaction reactor. However, stoichiometric ratios of approximately 1:4 are preferred. More preferably, the stoichiometric ratio of titanium tetrachloride to o-phthalodinitrile is about 1:4.2 and titanium trichloride to o-phthalodinitrile is about 1:3.42. Avoiding use of stoichiometric ratios other than the aforementioned ratios may be desirable, since lower or higher ratios may result in some disadvantages such as reduction in reaction yields, increase in side reactions and formation of byproducts although the objective of this invention may be attained.

Any organic solvent may be used in the synthesizing reaction. An organic solvent containing chlorine ions is preferred. More preferably, 1-chloronaphthalene is the organic solvent used in the process. Any organic solvent may be used in a quantity 2 to 4 times greater than the quantity of o-phthalodinitrile. A quantity out of this range will lower the yield and accordingly be economically disadvantageous, although the reaction may then proceed well. When 1-chloronaphthalene is selected as a solvent, the preferred stoichiometric ratio of titanium tetrachloride to 1-chloronaphthalene is about 1:11.1 and titanium trichloride to 1-chloronaphthalene is about 1:9.52.

The molecular sieve is alumino-silicates and has a mono-disperse and porous microstructure. The alumino-silicates may contain potassium, sodium and calcium so a strong ionic bond would be generated between alumino-silicates and polar molecules. Thus, the molecular sieve has a good moisture-absorbing ability. Another advantage of the molecular sieve is the molecular sieve will neither expand nor turn into solution after use for desiccant. Therefore, use of the molecular sieve in the process includes efficiently dehydrating the reaction reactor during the process to prevent side reactions. In addition, the molecular sieve has the advantages of being low cost and recyclable. Preferably, the molecular sieve used in the process is sodium calcium alumino-silicates having 3 Å, 4 Å or 5 Å pore diameter, and a molecular sieve with a 4 Å pore diameter is more preferred.

The weight ratio of titanium tetrachloride or titanium trichloride to the molecular sieve is from 1:0.5 to 1:5.0, and 1:5.0 is preferred. Lower ratios may increase side reactions and cause the reactants to be hard to react so the reaction yield is diminished. Larger ratios cause violent synthesizing reaction and increase the cost.

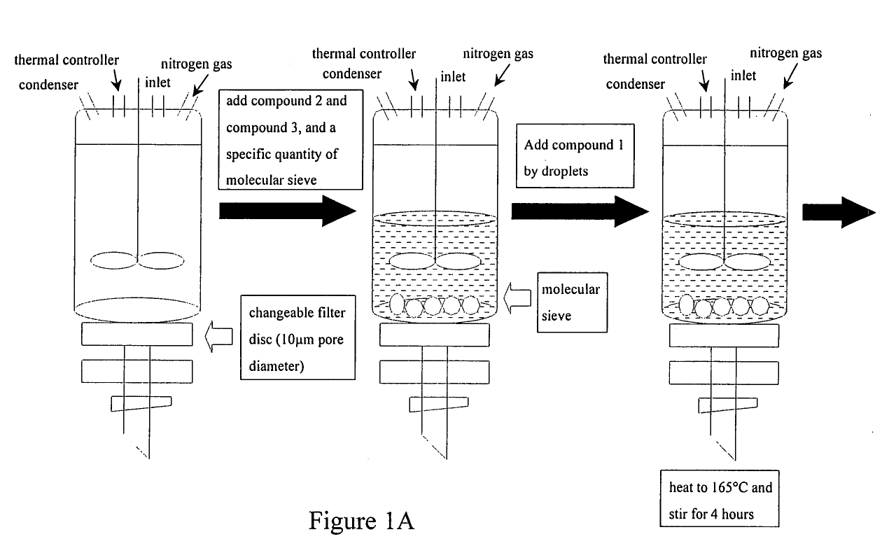

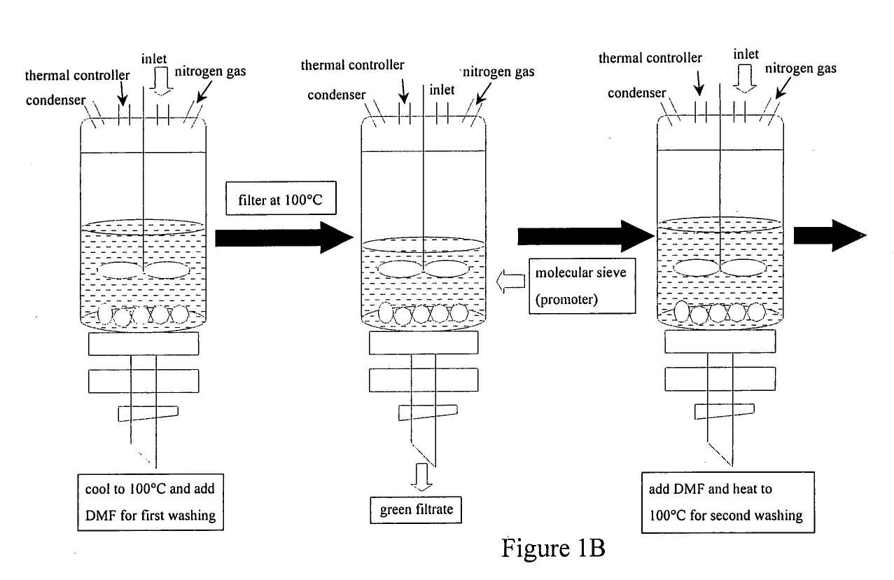

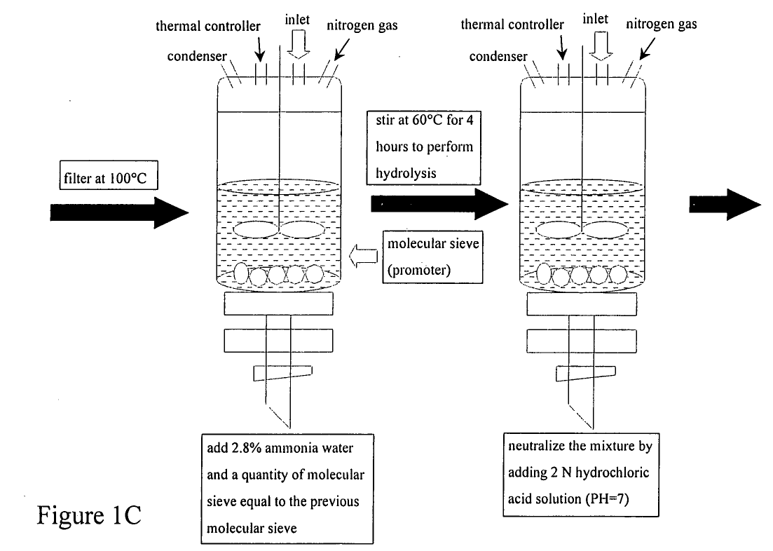

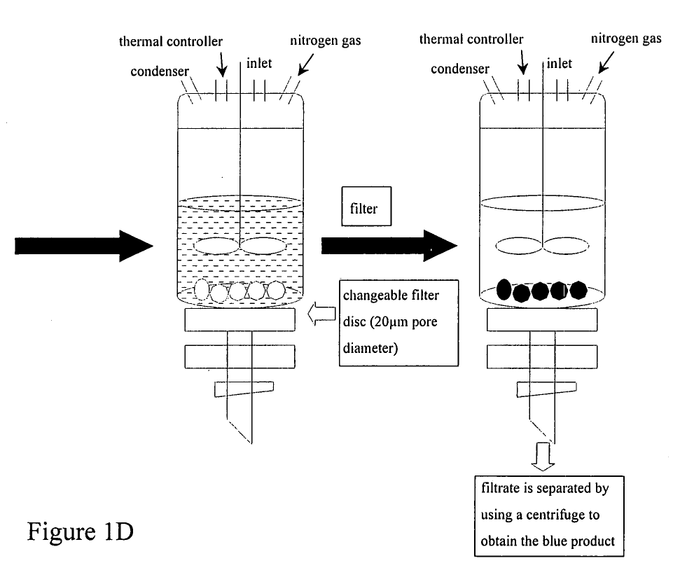

With reference to FIG. 1A, the molecular sieve promoter is added to a reaction reactor with an organic solvent and o-phthalodinitrile inside the reaction reactor. Then, titanium tetrachloride or titanium trichloride is added drop-wise to the reaction reactor, and the synthesizing reaction is performed. The mixture is heated to a temperature of from 105° C. to 165° C. and stirred for about 3 to 4 hours until the synthesizing reaction is finished. To maximize the effect of the molecular sieve as a promoter, the molecular sieve is divided into two equal portions. One portion is added before the synthesizing reaction, and the other portion is added after the synthesizing reaction is finished but before hydrolysis. The molecular sieve is used to dehydrate the reaction reactor during the synthesizing reaction and to accelerate the removal of chlorine ions from intermediate product, dichlorotitanium phthalocyanine, during hydrolysis.

With reference to FIGS. 1B and 1C, the intermediate product, dichlorotitanium phthalocyanine, is filtered at a temperature range of 40° C.-100° C. with an organic solvent after the synthesizing reaction of o-phthalodinitrile with titanium tetrachloride or titanium trichloride to purify and form a filter cake containing the intermediate product. The organic solvent used in filtering dichlorotitanium phthalocyanine may be selected from dimethyl formamide, dihalomethane, halobenzene and alcohol containing 1-5 carbons. Dichlorotitanium phthalocyanine can be filtered with filter paper, filter cloth, an changeable filter disc or a centrifuge. The changeable filter disc is polytetrafluoroethylene (i.e. Teflon) and has filtering pores. The filtering pores have a diameter selected from 5 μm to 100 μm.

With reference to FIGS. 1C and 1D, the filter cake containing the intermediate in the reaction reactor is hydrolyzed with ammonia water after the filtration process to form a solution. The solution is then neutralized by adding an acid such as hydrochloric acid, filtered, washed with deionized water and alcohol containing 1-5 carbons, and dried to obtain titanyl phthalocyanine. The molecular sieve in the reaction reactor can be recycled and used in the next cycle of the process.

In a preferred embodiment of the process for preparing titanyl phthalocyanine in accordance with the present invention the organic solvent is 1-chloronaphthalene solvent and is represented by the following scheme (I).

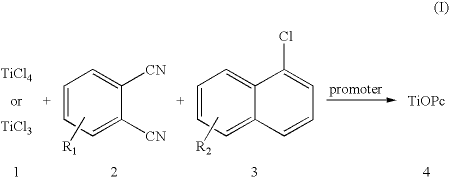

In the foregoing formula, some conditions are either required or preferred. First, R₁ and R₂ are independently selected from the group consisting of hydrogen, alkyl (C₁-C₅), alkoxy (C₁-C₅) and phenyl. In addition, a stoichiometric ratio of titanium tetrachloride: o-phthalodinitrile: 1-chloronaphthalene is 1:4.2±0.2:11.1±0.5 or titanium trichloride: o-phthalodinitrile: 1-chloronaphthalene is 1:3.42±0.2:9.52±0.5. The weight ratio of titanium tetrachloride (or titanium trichloride) to the molecular sieve is from 1:0.5 to 1:5.0.

The titanyl phthalocyanine obtained is used as a charge generating layer in photoconductors. The obtained titanyl phthalocyanine is a crystalline compound and needs to be powdered, for example, with a ball mill or a rotational gravel grinder to prepare a dispersive suspension. A well dispersive suspension is obtained by further ultrasonically emulsifying the suspension before application of the suspension as a charge generating layer and further testing photoelectric properties of a photoconductor.

The following examples describe the present invention in more detail to assist people with ordinary knowledge in the art in practicing the invention. However, the examples are not to be construed as limiting the scope of the invention.

### EXAMPLE 1

1.419 kg (11.07 mole) of ortho-phthalonitrile, 4.2 L (30.84 mole) of 1-chloronaphthalene and 250 g of 4 Å molecular sieve as a promoter (the molecular sieve UOP type 4 Å beads purchased from the Fluka company) were added in sequence to a 10 liter reaction reactor fitted with a mechanical stirrer, a thermometer, a thermal controller, a condenser and an exchangeable filter disc having a 10 μm pore diameter. The resulting mixture was stirred for 5 minutes, and 0.5 kg (2.64 mole) of titanium tetrachloride was added drop-wise over the next 10 minutes. The entire mixture was heated to 165° C. and stirred for 3 hours. Then, the mixture was cooled to 100° C., and 4 L of dimethyl formamide (DMF) was added by drops to the mixture. The resulting mixture was stirred for 20 minutes and filtered at 100° C. A green filtrate passed through the filter and was discarded. Another 4 L of dimethyl formamide was added to the mixture, and the mixture was heated to 100° C., stirred for 20 minutes and filtered. Green filtrate passing through the filter was discarded, and the resulting filter cake and promoter were held in the reaction reactor and cooled to 60° C. 3 L of a solution of 2.8% ammonia and water and 250 g of 4 Å molecular sieve as promoter were added to the reaction reactor and stirred for hydrolysis at 60° C. for 4 hours. Afterward, 50 mL of 2N hydrochloric acid solution was added to the reaction reactor to neutralize the mixture. Subsequently, the 10 μm pore filter disc was replaced by a 20 μm pore filter disc. Then, the reaction reactor was filled with nitrogen gas, and the solution was filtered through the 20 μm pore filter disc. The promoter remained in the reaction reactorl, and the filtrate was centrifuged to obtain a blue product. Purified titanyl phthalocyanine was obtained by washing the blue product twice with 2.5 L of deionized water, and drying the purified titanyl phthalocyanine at 100° C. The final product was weighted, and 1.43 kg of purified titanyl phthalocyanine was obtained at a yield of 90% (melting point 559° C.˜561° C.).

### EXAMPLE 2

1.419 kg (11.07 mole) of ortho-phthalonitrile, 4.2 L (30.84 mole) of 1-chloronaphthalene and 250 g of 3 Å molecular sieve as a promoter (the molecular sieve UOP type 3 Å beads purchased from the Fluka company) were added in sequence to a 10 liter reaction reactor fitted with a mechanical stirrer, a thermometer, a thermal controller, a condenser and an exchangeable filter disc having a 10 μm pore diameter. The resulting mixture was stirred for 5 minutes, and 0.5 kg (2.64 mole) of titanium tetrachloride was added drop-wise over the next 10 minutes. The entire mixture was heated to 165° C. and stirred for 3 hours. Then, the mixture was cooled to 100° C., and 4 L of dimethyl formamide (DMF) was added by drops to the mixture. The resulting mixture was stirred for 20 minutes and filtered at 100° C. A green filtrate passed through the filter and was discarded. Another 4 L of dimethyl formamide was added to the mixture, and the mixture was heated to 100° C., stirred for 20 minutes and filtered. Green filtrate passing through the filter was discarded, and the resulting filter cake and promoter were held in the reaction reactor and cooled to 60° C. 3 L of a solution of 2.8% ammonia and water and 250 g of 3 Å molecular sieve as promoter were added to the reaction reactor and stirred for hydrolysis at 60° C. for 4 hours. Afterward, 50 mL of 2N hydrochloric acid solution was added to the reaction reactor to neutralize the mixture. Subsequently, the 10 μm pore filter disc was replaced by a 20 μm pore filter disc. Then, the reaction reactor was filled with nitrogen gas, and the solution was filtered through the 20 μm pore filter disc. The promoter remained in the reaction reactor, and the filtrate was centrifuged to obtain a blue product. Purified titanyl phthalocyanine was obtained by washing the blue product twice with 2.5 L of deionized water, and drying the purified titanyl phthalocyanine at 100° C. The final product was weighted, and 1.28 kg of purified titanyl phthalocyanine was obtained at a yield of 80% (melting point 559° C.˜561° C.).

### EXAMPLE 3

1.419 kg (11.07 mole) of ortho-phthalonitrile, 4.2 L (30.84 mole) of 1-chloronaphthalene and 250 g of 5 Å molecular sieve as a promoter (the molecular sieve UOP type 5 Å beads purchased from the Fluka company) were added in sequence to a 10 liter reaction reactor fitted with a mechanical stirrer, a thermometer, a thermal controller, a condenser and an exchangeable filter disc having a 10 μm pore diameter. The resulting mixture was stirred for 5 minutes, and 0.5 kg (2.64 mole) of titanium tetrachloride was added drop-wise over the next 10 minutes. The entire mixture was heated to 165° C. and stirred for 3 hours. Then, the mixture was cooled to 100° C., and 4 L of dimethyl formamide (DMF) was added by drops to the mixture. The resulting mixture was stirred for 20 minutes and filtered at 100° C. A green filtrate passed through the filter and was discarded. Another 4 L of dimethyl formamide was added to the mixture, and the mixture was heated to 100° C., stirred for 20 minutes and filtered. Green filtrate passing through the filter was discarded, and the resulting filter cake and promoter were held in the reaction reactor and cooled to 60° C. 3 L of a solution of 2.8% ammonia and water and 250 g of 5 Å molecular sieve as promoter were added to the reaction reactor and stirred for hydrolysis at 60° C. for 4 hours. Afterward, 50 L of 2N hydrochloric acid solution was added to the reaction reactor to neutralize the mixture. Subsequently, the 10 μm pore filter disc was replaced by a 20 μm pore filter disc. Then, the reaction reactor was filled with nitrogen gas, and the solution was filtered through the 20 μm pore filter disc. The promoter remained in the reaction reactor, and the filtrate was centrifuged to obtain a blue product. Purified titanyl phthalocyanine was obtained by washing the blue product twice with 2.5 L of deionized water, and drying the purified titanyl phthalocyanine at 100° C. The final product was weighted, and 1.2 kg of purified titanyl phthalocyanine was obtained at a yield of 75% (melting point 559° C.˜561° C.).

### EXAMPLE 4

1.419 kg (11.07 mole) of ortho-phthalonitrile, 4.2 L (30.84 mole) of 1-chloronaphthalene and 250 g of 4 Å molecular sieve as a promoter (the molecular sieve UOP type 4 Å beads purchased from the Fluka company) were added in sequence to a 10 liter reaction reactor fitted with a mechanical stirrer, a thermometer, a thermal controller, a condenser and an exchangeable filter disc having a 10 μm pore diameter. The resulting mixture was stirred for 5 minutes, and 0.5 kg (2.64 mole) of titanium tetrachloride was added drop-wise over the next 10 minutes. The entire mixture was heated to 165° C. and stirred for 3 hours. Then, the mixture was cooled to 25° C., and 4 L of dichloromethane was added by drops to the mixture. The resulting mixture was stirred for 20 minutes and filtered at 40° C. A green filtrate passed through the filter and was discarded. Another 4 L of dichloromethane was added to the mixture, and the mixture was heated to 40° C., stirred for 20 minutes and filtered. Green filtrate passing through the filter was discarded, and the resulting filter cake and promoter were held in the reaction reactor and cooled to 25° C. 3 L of a solution of 2.8% ammonia and water and 250 g of 4 Å molecular sieve as promoter were added to the reaction reactor and stirred for hydrolysis at 60° C. for 4 hours. Afterward, 50 mL of 2N hydrochloric acid solution was added to the reaction reactor to neutralize the mixture. Subsequently, the 10 μm pore filter disc was replaced by a 20 μm pore filter disc. Then, the reaction reactor was filled with nitrogen gas, and the solution was filtered through the 20 μm pore filter disc. The promoter remained in the reaction reactor, and the filtrate was centrifuged to obtain a blue product. Purified titanyl phthalocyanine was obtained by washing the blue product twice with 2.5 L of deionized water, and drying the purified titanyl phthalocyanine at 100° C. The final product was weighted, and 1.25 kg of purified titanyl phthalocyanine was obtained at a yield of 78% (melting point 559° C.˜561° C.).

### EXAMPLE 5

1.419 kg (11.07 mole) of ortho-phthalonitrile, 4.2 L (30.84 mole) of 1-chloronaphthalene and 250 g of 4 Å molecular sieve as a promoter (the molecular sieve UOP type 4 Å beads purchased from the Fluka company) were added in sequence to a 10 liter reaction reactor fitted with a mechanical stirrer, a thermometer, a thermal controller, a condenser and an exchangeable filter disc having a 10 μm pore diameter. The resulting mixture was stirred for 5 minutes, and 0.5 kg (3.24 mole) of titanium trichloride was added drop-wise over the next 10 minutes. The entire mixture was heated to 165° C. and stirred for 3.5 hours. Then, the mixture was cooled to 100° C., and 4 L of dimethyl formamide (DMF) was added by drops to the mixture. The resulting mixture was stirred for 20 minutes and filtered at 100° C. A green filtrate passed through the filter and was discarded. Another 4 L of dimethyl formamide was added to the mixture, and the mixture was heated to 100° C., stirred for 20 minutes and filtered. Green filtrate passing through the filter was discarded, and the resulting filter cake and promoter were held in the reaction reactor and cooled to 60° C. 3 L of a solution of 2.8% ammonia and water and 250 g of 4 Å molecular sieve as promoter were added to the reaction reactor and stirred for hydrolysis at 60° C. for 4 hours. Afterward, 40 mL of 2N hydrochloric acid solution was added to the reaction reactor to neutralize the mixture. Subsequently, the 10 μm pore filter disc was replaced by a 20 μm pore filter disc. Then the reaction reactor was filled with nitrogen gas, and the solution was filtered through the 20 μm pore filter disc. The promoter remained in the reaction reactor, and the filtrate was centrifuged to obtain a blue product. Purified titanyl phthalocyanine was obtained by washing the blue product twice with 2 L of deionized water, and drying the purified titanyl phthalocyanine at 100° C. The final product was weighted, and 1.39 kg of purified titanyl phthalocyanine was-obtained at a yield of 88% (melting point 559° C.˜561° C.).

### EXAMPLE 6

1.419 kg (11.07 mole) of ortho-phthalonitrile, 4.2 L (30.84 mole) of 1-chloronaphthalene and 250 g of 3 Å molecular sieve as a promoter (the molecular sieve UOP type 3 Å beads purchased from the Fluka company) were added in sequence to a 10 liter reaction reactor fitted with a mechanical stirrer, a thermometer, a thermal controller, a condenser and an exchangeable filter disc having a 10 μm pore diameter. The resulting mixture was stirred for 5 minutes, and 0.5 kg (3.24 mole) of titanium trichloride was added drop-wise over the next 10 minutes. The entire mixture was heated to 165° C. and stirred for 3.5 hours. Then, the mixture was cooled to 100° C., and 4 L of dimethyl formamide (DMF) was added by drops to the mixture. The resulting mixture was stirred for 20 minutes and filtered at 100° C. A green filtrate passed through the filter and was discarded. Another 4 L of dimethyl formamide was added to the mixture, and the mixture was heated to 100° C., stirred for 20 minutes and filtered. Green filtrate passing through the filter was discarded, and the resulting filter cake and promoter were held in the reaction reactor and cooled to 60° C. 3 L of a solution of 2.8% ammonia and water and 250 g of 3 Å molecular sieve as promoter were added to the reaction reactor and stirred for hydrolysis at 60° C. for 4 hours. Afterward, 40 mL of 2N hydrochloric acid solution was added to the reaction reactor to neutralize the mixture. Subsequently, the 10 μm pore filter disc was replaced by a 20 μm pore filter disc. Then the reaction reactor was filled with nitrogen gas, and the solution was filtered through the 20 μm pore filter disc. The promoter remained in the reaction reactor, and the filtrate was centrifuged to obtain a blue product. Purified titanyl phthalocyanine was obtained by washing the blue product twice with 2 L of deionized water, and drying the purified titanyl phthalocyanine at 100° C. The final product was weighted, and 1.29 kg of purified titanyl phthalocyanine was obtained at a yield of 81% (melting point 559° C.˜561° C.).

### EXAMPLE 7

1.419 kg (11.07 mole) of ortho-phthalonitrile, 4.2 L (30.84 mole) of 1-chloronaphthalene and 250 g of 5 Å molecular sieve as a promoter (the molecular sieve UOP type 5 Å beads purchased from the Fluka company) were added in sequence to a 10 liter reaction reactor fitted with a mechanical stirrer, a thermometer, a thermal controller, a condenser and an exchangeable filter disc having a 10 μm pore diameter. The resulting mixture was stirred for 5 minutes, and 0.5 kg (3.24 mole) of titanium trichloride was added drop-wise over the next 10 minutes. The entire mixture was heated to 165° C. and stirred for 3.5 hours. Then, the mixture was cooled to 100° C., and 4 L of dimethyl formamide (DMF) was added by drops to the mixture. The resulting mixture was stirred for 20 minutes and filtered at 100° C. A green filtrate passed through the filter and was discarded. Another 4 L of dimethyl formamide was added to the mixture, and the mixture was heated to 100° C., stirred for 20 minutes and filtered. Green filtrate passing through the filter was discarded, and the resulting filter cake and promoter were held in the reaction reactor and cooled to 60° C. 3 L of a solution of 2.8% ammonia and water and 250 g of 5 Å molecular sieve as promoter were added to the reaction reactor and stirred for hydrolysis at 60° C. for 4 hours. Afterward, 40 mL of 2N hydrochloric acid solution was added to the reaction reactor to neutralize the mixture. Subsequently, the 10 μm pore filter disc was replaced by a 20 μm pore filter disc. Then the reaction reactor was filled with nitrogen gas, and the solution was filtered through the 20 μm pore filter disc. The promoter remained in the reaction reactor, and the filtrate was centrifuged to obtain a blue product. Purified titanyl phthalocyanine was obtained by washing the blue product twice with 2 L of deionized water, and drying the purified titanyl phthalocyanine at 100° C. The final product was weighted, and 1.18 kg of purified titanyl phthalocyanine was obtained at a yield of 74% (melting point 559° C.-561° C.).

### EXAMPLE 8

1.419 kg (11.07 mole) of ortho-phthalonitrile, 4.2 L (30.84 mole) of 1-chloronaphthalene and 1.25 kg of 4 Å molecular sieve as a promoter (the molecular sieve UOP type 4 Å beads purchased from the Fluka company) were added in sequence to a 10 liter reaction reactor fitted with a mechanical stirrer, a thermometer, a thermal controller, a condenser and an exchangeable filter disc having a 10 μm pore diameter. The resulting mixture was stirred for 5 minutes, and 0.5 kg (2.64 mole) of titanium tetrachloride was added drop-wise over the next 10 minutes. The entire mixture was heated to 165° C. and stirred for 3 hours. Then, the mixture was cooled to 100° C., and 5 L of dimethyl formamide (DMF) was added by drops to the mixture. The resulting mixture was stirred for 20 minutes and filtered at 100° C. A green filtrate passed through the filter and was discarded. Another 5 L of dimethyl formamide was added to the mixture, and the mixture was heated to 100° C., stirred for 20 minutes and filtered. Green filtrate passing through the filter was discarded, and the resulting filter cake and promoter were held in the reaction reactor and cooled to 60° C. 3 L of a solution of 2.8% ammonia and water and 1.25 kg of 4 Å molecular sieve as promoter were added to the reaction reactor and stirred for hydrolysis at 60° C. for 4 hours. Afterward, 50 mL of 2N hydrochloric acid solution was added to the reaction reactor to neutralize the mixture. Subsequently, the form pore filter disc was replaced by a 20 μm pore filter disc. Then, the reaction reactor was filled with nitrogen gas, and the solution was filtered through the 20 μm pore filter disc. The promoter remained in the reaction reactor, and the filtrate was centrifuged to obtain a blue product. Purified titanyl phthalocyanine was obtained by washing the blue product twice with 2.5 L of deionized water, and drying the purified titanyl phthalocyanine at 100° C. The final product was weighted, and 1.43 kg of purified titanyl phthalocyanine was obtained at a yield of 90% (melting point 559° C.˜561° C.).

### EXAMPLE 9

1.4 kg (11 mole) of ortho-phthalonitrile, 4.2 L (30.84 mole) of 1-chloronaphthalene, and 2 kg of n-amyl alcohol as a promoter were added in sequence to a 10 liter reaction reactor fitted with a mechanical stirrer, a thermometer, a thermal controller, a condenser and an exchangeable filter disc having a 10 μm pore diameter. The resulting mixture was stirred for 5 minutes, and 0.5 kg (2.64 mole) of titanium tetrachloride was added drop-wise over the next 10 minutes. The entire mixture was heated to 165° C. and stirred for 3 hours. Then, the mixture was cooled to 100° C., and 5 L of dimethyl formamide (DMF) was added by drops to the mixture. The resulting mixture was stirred for 20 minutes and filtered at 100° C. A green filtrate passed through the filter and was discarded. Another 5 L of dimethyl formamide was added to the mixture, and the mixture was heated to 100° C., stirred for 20 minutes and filtered. Green filtrate passing through the filter was discarded, and the resulting filter cake and promoter were held in the reaction reactor and cooled to 60° C. 3 L of a solution of 2.8% ammonia and water was added to the reaction reactor and stirred for hydrolysis at 60° C. for 8 hours. Afterward, 60 mL of 2N hydrochloric acid solution was added to the reaction reactor to neutralize the mixture. Subsequently, the 10 μm pore filter disc was replaced by a 20 μm pore filter disc. Then, the reaction reactor was filled with nitrogen gas, and the solution was filtered through the 20 μm pore filter disc. The promoter remained in the reaction reactor, and the filtrate was centrifuged to obtain a blue product. Purified titanyl phthalocyanine was obtained by washing the blue product four times with 2.5 L of deionized water, and drying the purified titanyl phthalocyanine at 100° C. The final product was weighted, and 1.1 kg of purified titanyl phthalocyanine was obtained at a yield of 70% (melting point 564° C.˜565° C.).

### EXAMPLE 10

1.42 kg (11.07 mole) of ortho-phthalonitrile and 4.2 L (30.84 mole) of 1-chloronaphthalene were added in sequence to a 10 liter reaction reactor fitted with a mechanical stirrer, a thermometer, a thermal controller, a condenser and an exchangeable filter disc having a 10 μm pore diameter. The resulting mixture was stirred for 5 minutes, and 0.5 kg (2.64 mole) of titanium tetrachloride was added drop-wise over the next 10 minutes. The entire mixture was heated to 165° C. and stirred for 3 hours. Then, the mixture was cooled to 100° C., and 4 L of dimethyl formamide (DMF) was added by drops to the mixture. The resulting mixture was stirred for 20 minutes and filtered at 100° C. A green filtrate passed through the filter and was discarded. Another 5 L of dimethyl formamide was added to the mixture, and the mixture was heated to 100° C., stirred for 20 minutes and filtered. Green filtrate passing through the filter was discarded, and the resulting filter cake and promoter were held in the reaction reactor and cooled to 60° C. 5 L of a solution of 2.8% ammonia and water and 500 g (5.3 mole) of phenol were added to the reaction reactor and stirred for hydrolysis at 60° C. for 10 hours. Afterward, 500 mL of 2N hydrochloric acid solution was added to the reaction reactor to neutralize the mixture. Subsequently, the 10 μm pore filter disc was replaced by a 20 μm pore filter disc. Then, the reaction reactor was filled with nitrogen gas, and the solution was filtered through the 20 μm pore filter disc. The promoter remained in the reaction reactor, and the filtrate was centrifuged to obtain a blue product. Purified titanyl phthalocyanine was obtained by washing the blue product seven times with 2.5 L of deionized water, and drying the purified titanyl phthalocyanine at 100° C. The final product was weighted, and 0.97 kg of purified titanyl phthalocyanine was obtained at a yield of 61% (melting point 570° C.˜571° C.).

<Photoelectric Properties Tests>

To perform a test for photoelectric properties, a charge generating layer was applied to an aluminum substrate by a dip method. Then a charge transport layer was applied to the charge generating layer to achieve an organic photoconductor drum.

The charge generating layers were composed of 50% of polyvinylbutyral and 50% titanyl phthalocyanine prepared respectively in examples 1-10 and powdered with a ball mill in accordance with the present invention.

The material used to form the charge transport layer originally was a solution and was prepared by mixing 40 wt % of benzidine compounds mixture (TPD: N,N′-Bis-(3-methylphenyl)-N,N′-bis-(phenyl)-benzidine (86%±3%), N,N,N′-Tris-(phenyl)-N′-(m-tolyl)-benzidine(13%±2%) and N,N,N′,N′-Tetraphenylbenzidine(0.2%-1.5%)) and 60 wt % of polycarbonate-A (based on the weight of the charge transport layer) in a composite solvent composed of dichloromethane and toluene.

<Photoelectric Properties>

The organic photoconductor drums prepared were tested by using PDT-2000LA (QEA Inc. SN: 02021501070217), and the photoelectric properties observed are listed in Table 1.

TABLE 1

| Examples | V₀ (volts) | Vᵣ (volts) | E_{1/2} (μJ/cm²) | DₖD_{ec} (%) |
|---|---|---|---|---|
| HP-4100 | 706.28 | 54.47 | 0.070 | 97.1 |
| 1 | 704.35 | 32.30 | 0.090 | 97.9 |
| 2 | 697.13 | 24.90 | 0.084 | 95.9 |
| 3 | 704.43 | 33.61 | 0.083 | 96.9 |
| 4 | 694.59 | 26.29 | 0.084 | 95.8 |
| 5 | 702.35 | 35.30 | 0.090 | 97.9 |
| 6 | 698.13 | 34.90 | 0.084 | 95.9 |
| 7 | 702.43 | 23.61 | 0.083 | 96.9 |
| 8 | 699.59 | 26.29 | 0.084 | 95.8 |
| 9 | 723.36 | 46.57 | 0.084 | 97.9 |
| 10 | 726.69 | 43.00 | 0.084 | 98.4 |

V₀: initial surface potential
Vᵣ: residual potential (measured at a moment of six times of half-life)
E_{1/2}: sensitivity (the intensity of the light required to reduce the surface potential of the drum to half of the initial surface potential of the drum)
DₖD_{ec}: dark decay

The photoelectric properties of organic photoconductor drums using titanyl phthalocynines prepared in examples 1-10 in accordance with the present invention and the organic photoconductor drum produced by Hewlett Packard using TiOPc prepared conventionally as the charge generating layer and TPD used as the charge transport layer in the present invention are listed in Table 1. All organic photoconductor drums in Table 1 conformed with the following photoelectric requirements:

V₀ (initial surface potential)>670 volt

Vᵣ (residual potential)<60 volt

E_{1/2} (sensitivity): 0.1±0.02 μj/cm²

DₖD_{ec} (dark decay)>95%

The results recorded in Table 1 clearly show that the organic photoconductor drums coated with titanyl phthalocyanines in accordance with the present invention have lower residual potential than HP's organic photoconductor drum.

The present invention provides a process for preparing titanyl phthalocyanine that carries out a synthesizing reaction and hydrolysis in the same reaction reactor without separating an intermediate product in the presence of a molecular sieve as a promoter so the operation time is decreased, the process is simplified, and the manufacturing cost is reduced. In addition, the molecular sieve (porous sodium calcium alumino-silicate) used as a promoter can dehydrate and accelerate the chlorine ions removal from dichlorotitanium phthalocyanine so high-purity titanyl phthalocyanine can be obtained in a short time compared to conventional processes. As seen from Table 1 above, the photoconductor drum using the high-purity titanyl phthalocyanine obtained from the process in this invention as charge generating layer has quite excellent photoelectric properties so the titanyl phthalocyanine prepared in accordance with the present invention is especially useful as a charge generating material for photocoductor drums.

Although the invention has been explained in relation to its preferred embodiment, many other possible modifications and variations can be made without departing from the spirit and scope of the invention as hereinafter claimed.

What is claimed is:

1. A process for preparing titanyl phthalocyanine comprising effecting a synthesizing reaction of titanium tetrachloride or titanium trichloride with o-phthalodinitrile in an organic solvent in the presence of a molecular sieve as a promoter for about 3 to 4 hours to obtain dichlorotitanium phthalocyanine, and hydrolyzing the resulting dichlorotitanium phthalocyanine.

2. The process according to claim 1, wherein a stoichiometric ratio of titanium tetrachloride to o-phthalodinitrile is about 1:4.2.

3. The process according to claim 1, wherein a stoichiometric ratio of titanium trichloride to o-phthalodinitrile is about 1:3.42.

4. The process according to claim 1, wherein the organic solvent is used in a quantity 2 to 4 times greater than the quantity of o-phthalodinitrile.

5. The process according to claim 1, wherein the organic solvent is 1-chloronaphthalene, and a stoichiometric ratio of titanium tetrachloride to 1-chloronaphthalene is about 1:11.1.

6. The process according to claim 1, wherein the organic solvent is 1-chloronaphthalene, and a stoichiometric ratio of titanium trichloride to 1-chloronaphthalene is about 1:9.52.

7. The process according to claim 1, wherein the molecular sieve is porous sodium calcium alumino-silicate having a pore diameter selected from a group having 3 Å, 4 Å or 5 Å.

8. The process according to claim 1, wherein the weight ratio of titanium tetrachloride or titanium trichloride to the molecular sieve is from 1:0.5 to 1:5.0.

9. The process according to claim 8, wherein the molecular sieve is divided into two equal portions of which one portion is added before the synthesizing reaction and the other portion is added after the synthesizing reaction is finished but before the hydrolysis.

10. The process according to claim 1, wherein the resulting dichlorotitanium phthalocyanine is further filtered at a temperature range of 40-100° C. before hydrolysis.

11. The process according to claim 10, wherein a solvent used in filtering is selected from dimethyl formamide, dihalomethane, halobenzene and alcohol containing 1-5 carbons.

12. The process according to claim 10, wherein the resulting dichlorotitanium phthalocyanine is filtered with filter paper, filter cloth, an changeable filter disc or a centrifuge.

13. The process according to claim 12, wherein the changeable filter disc has a pore diameter from 5 μm to 100 μm.

14. The process according to claim 12, wherein the changeable filter disc is polytetrafluoroethylene (i.e. Teflon).

15. A process for preparing titanyl phthalocyanine (4) comprising effecting a synthesizing reaction of titanium tetrachloride or titanium trichloride (1) with o-phthalodinitrile (2) in a 1-chloronaphthalene solvent (3) in the presence of a molecular sieve as a promoter for about 3 to 4 hours resulting in dichlorotitanium phthalocyanine, filtering the resulting dichlorotitanium phthalocyanine adding another equal portion of molecular sieve as additional promoter and hydrolyzing the dichlorotitanium phthalocyanine, as shown in the following scheme (I): 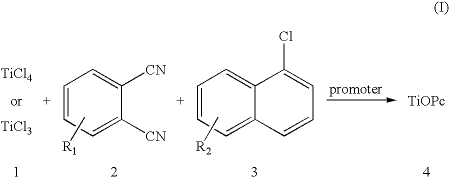
wherein
R₁ and R₂ are independently selected from a group consisting of hydrogen, alkyl (C₁-C₅), alkoxy (C₁-C₅) and phenyl;
a stoichiometric ratio of titanium tetrachloride (1): o-phthalodinitrile (2): 1-chloronaphthalene (3) is 1:4.2±0.2:11.1±0.5 or titanium trichloride (1): o-phthalodinitrile (2): 1-chloronaphthalene (3) is 1:3.42±0.2:9.52±0.5; and
the weight ratio of the titanium tetrachloride or titanium trichloride to the molecular sieve is from 1:0.5 to 1:5.0.
